(19) United States
(12) Patent Application Publication
(10) Pub. No.: US 2006/0182689 A1
(43) Pub. Date: Aug. 17, 2006

(54) Process for separating out at least one organic compound

(76) Inventors: Pierre Dournel, Brussels (BE); Michel Surbled, Saint Nolff (FR); Bernard Mompon, Vannes (FR)

Correspondence Address: CONNOLLY BOVE LODGE & HUTZ, LLP, P O BOX 2207, WILMINGTON, DE, 19899, US

(21) Appl. No.: 10/557,314

(22) PCT Filed: May 19, 2004
(86) PCT No.: PCT/EP04/50870

§ 371 Date: Feb. 24, 2006

(30) Foreign Application Priority Data
May 22, 2003 (FR) 03 06201

(51) Int. Cl. A61L 9/04; A61K 8/00; C07C 405/00; C07J 9/00; C07C 61/06

(52) U.S. Cl. 424/45; 424/47; 552/540; 562/503

(57) ABSTRACT

Process for separating out at least one organic compound from a substrate containing the said organic compound, comprising a treatment of the substrate with an extraction medium comprising at least one fluoro compound with an atmospheric boiling point of greater than or equal to 25° C., so as to form a fraction comprising organic compound and fluoro compound.

The present invention relates to a process for separating out at least one organic compound.

During the preparation of medicinal products or cosmetic products, organic compounds or compositions of organic compounds of natural origin are often used, for example as active principle. Generally, natural raw materials contain desired organic compounds in low concentration in a substrate.

It is consequently necessary to isolate the organic compounds or the compositions of organic compounds from the substrate.

Patent application NO-A-00/64555 describes the extraction of active principles from a raw material with a solvent containing pentafluoropropane. The handling of this solvent and of fractions containing the active principle is difficult and the extraction selectivity is not always satisfactory.

The invention is directed towards overcoming these problems.

The invention consequently relates to a process for separating out at least one organic compound from a substrate containing the said organic compound, comprising a treatment of the substrate with an extraction medium comprising at least one fluoro compound with an atmospheric boiling point of greater than or equal to 25° C., so as to form a fraction comprising organic compound and fluoro compound.

It has been found, surprisingly, that the process according to the invention allows an efficient and selective separation of desired organic compounds from a substrate, in particular of natural origin. The organic compounds may be recovered essentially without undergoing chemical modifications. The process according to the invention may be performed easily.

In the process according to the invention, the fluoro compound has a boiling point at 101.3 kPa of greater than or equal to about 25° C. Good results are obtained with a fluoro compound that has a boiling point at 101.3 kPa of greater than or equal to about 30° C. In one preferred variant, the fluoro compound has a boiling point at 101.3 kPa of greater than or equal to about 35° C. A fluoro compound with a boiling point at 101.3 kPa of greater than or equal to about 40° C. is most particularly preferred. The fluoro compound generally has a boiling point at 101.3 kPa of less than or equal to about 200° C. Good results are obtained with a fluoro compound that has a boiling point at 101.3 kPa of less than or equal to about 100° C. In one preferred variant, the fluoro compound has a boiling point at 101.3 kPa of less than or equal to 80° C.

In the process according to the invention, the fluoro compound generally has a Kauri-butanol number (ASTM D1133-02) of greater than or equal to about 0. Good results are obtained with a fluoro compound that has a Kauri-butanol number of greater than or equal to about 5. In one preferred variant, the fluoro compound has a Kauri-butanol number of greater than or equal to about 9. The fluoro compound generally has a Kauri-butanol number of less than or equal to about 50. Good results are obtained with a fluoro compound that has a Kauri-butanol number of less than or equal to about 30. In one preferred variant, the fluoro compound has a Kauri-butanol number of less than or equal to 20.

In the process according to the invention, the fluoro compound often contains only fluorine as halogen. It is preferably chosen from fluoro ethers, hydrofluoroalkanes and perfluoroalkanes.

The hydrofluorocarbons (HFC) and perfluorocabons that may be used in the process according to the invention may be linear, branched or cyclic and generally contain 4, 5, 6, 7, 8, 9 or 10 carbon atoms.

Typical examples of hydrofluoroalkanes are chosen from hydrofluorobutanes and hydrofluoropentanes. Specific examples of such hydrofluoroalkanes are 1,1,1,3,3-pentafluorobutane (HFC-365mfc) and 1,1,1,2,3,4,4,5,5,5-decafluoropentane (HFC-43-10mee). 1,1,1,3,3-Pentafluorobutane is most particularly preferred.

Among the perfluorocarbons, those comprising at least five carbon atoms are particularly suitable. Perfluoropentane and perfluorohexane are preferred. Perfluoropentane and perfluorohexane are often used in the form of technical mixtures of isomers, as sold, for example, by 3M under the respective names PF5050 for perfluoropentane and PF5060 for perfluorohexane. Perfluorohexane is particularly preferred.

The fluoro ethers that may be used in the process according to the invention may be linear, branched or cyclic and generally contain 3, 4, 5, 6, 7, 8, 9 or 10 carbon atoms. Among the fluoro ethers, those containing at least 4 carbon atoms are suitable for use. Perfluorobutyl methyl ether and perfluorobutyl ethyl ether are preferred. Perfluorobutyl methyl ether is particularly preferred.

In one variant, the fluoro ether is a perfluoropolyether. For the purposes of the present invention, the term “perfluoropolyether” is intended to denote a compound consisting essentially of carbon, fluorine and oxygen atoms and comprising at least 2 and preferably at least 3 ether bonds C—O—C, or a mixture of several compounds corresponding to this definition. Often, the oxygen atoms in the perfluoropolyether are exclusively present in ether bonds C—O—C.

Perfluoropolyethers that may be used are, for example, those corresponding to the general formulae CF3-[(OCF(CF3)-CF2)a-(O—CF2)b]O—CF3 (I) and CF3-[(OCF2-CF2)c-(O—CT2)d]O—CF3 (II) in which a, b, c and d independently denote integers greater than 0. Perfluoropolyethers that may be used are, for example, those sold by Solvay Solexis under the name Galden®.

In another variant, the fluoro ether is a hydrofluoropolyether. For the purposes of the present invention, the term “hydrofluoropolyether” is intended to denote a compound consisting essentially of carbon, fluorine, oxygen and hydrogen atoms, which contains at least one C—H bond and comprises at least two and preferably at least three ether bonds C—O—C, or a mixture of several compounds corresponding to this definition. Often, the oxygen atoms in the perfluoropolyether are exclusively present in ether bonds C—O—C. Generally, the hydrofluoropolyether contains a plurality of C—H bonds. Specific examples of hydrofluoropolyethers include at least one group —CF₂H. Hydrofluoropolyethers that may be used are, for example, those sold by Solvay Solexis under the name H-Galden®.

In the process according to the invention, the extraction medium often also comprises a non-fluoro cosolvent.

Examples of non-fluoro organic solvents that are suitable for use include hydrocarbons, chlorinated hydrocarbons, alcohols, esters, ketones and ethers.

The hydrocarbons that may be used in the process according to the invention may be linear, branched or cyclic and generally contain 3, 4, 5, 6, 7, 8, 9, 10, 11 or 12 carbon atoms. Hydrocarbons containing at least 5 carbon atoms are suitable for use. Preferably, the hydrocarbons contain at least 6 carbon atoms. Among the alkanes or alkenes, compounds containing from 5 to 12 carbon atoms are preferred. Pentanes, hexanes, heptanes or octanes are suitable for use. n-Hexane is particularly preferred.

n-Hexane has the property of forming azeotropic or pseudo-azeotropic mixtures with 1,1,1,3,3-pentafluorobutane, which may present advantages for certain applications. Azeotropic or pseudo-azeotropic mixtures are described in patent U.S. Pat. No. 6,303,668 in the name of the Applicant.

Among the aromatic hydrocarbons that are preferred are those comprising at least one alkyl substituent on a benzene nucleus. Toluene, 1,2-xylene, 1,3-xylene and 1,4-xylene or mixtures thereof are most particularly preferred.

The chlorinated hydrocarbons that may be used in the process according to the invention may be linear, branched or cyclic and general contain 1, 2, 3, 4, 5, 6, 7, 8, 9 or 10 carbon atoms. Chlorinated hydrocarbons containing 1,2,3 or 4 carbon atoms are suitable for use. Preferably, the chlorinated hydrocarbons contain one or two carbon atoms. Among the chloroalkanes, dichloromethane, trichloromethane and 1,2-dichloroethane are preferred. Among the chloro-alkenes, perchloroethylene and 1,2-dichloroethylene are preferred. trans-1,2-Dichloroethylene is most particularly preferred.

1,2-Dichloroethylene has the property of forming azeotropic or pseudo-azeotropic mixtures with 1,1,1,3,3-pentafluorobutane, which may present advantages for certain applications. Azeotropic or pseudo-azeotropic mixtures and ternary azeotropic or pseudo-azeotropic mixtures also comprising an alcohol are described in patent U.S. Pat. No. 5,478,492 in the name of the Applicant

The alcohols that may be used in the process according to the invention may be linear, branched or cyclic and generally contain 1, 2, 3, 4, 5, 6, 7, 8, 9 or 10 carbon atoms. Alcohols containing 1, 2, 3, 4 or 5 carbon atoms are suitable for use. Preferably, the alcohols contain 1, 2, 3 or 4 carbon atoms. Among the alkanols, methanol, ethanol, n-propanol, isopropanol, n-butanol, isobutanol and tert-butanol are preferred. Methanol, ethanol, isopropanol and isobutanol give good results. Isobutanol is most particularly preferred.

Methanol has the property of forming azeotropic or pseudo-azeotropic mixtures with 1,1,1,3,3-pentafluorobutane, which may present advantages for certain applications. The azeotropic or pseudo-azeotropic mixtures contain from 93% to 99% by weight of 1,1,1,3,3-pentafluorobutane and from 1% to 7% of methanol. The true azeotrope contains about 96.2% by weight of 1,1,1,3,3-pentafluorobutane and about 3.8% by weight of methanol.

Ethanol has the property of forming azeotropic or pseudo-azeotropic mixtures with 1,1,1,3,3-pentafluorobutane, which may present advantages for certain applications. The azeotropic or pseudo-azeotropic mixtures are described in patent U.S. Pat. No. 5,445,757 in the name of the Applicant.

The esters that may be used in the process according to the invention may be linear, branched or cyclic and generally contain 2, 3, 4, 5, 6, 7, 8, 9 or 10 carbon atoms. Esters containing 4, 5, 6, 7, 8 or 9 carbon atoms are suitable for use. Preferably, the esters are derivatives of a carboxylic acid containing at least two carbon atoms. Preferably, the esters are derivatives of an alkanol selected from the group consisting of methanol, ethanol, n-propanol, isopropanol n-butanol, isobutanol and tert-butanol. Ethyl acetate, ethyl butyrate and ethyl caproate are suitable for use.

The ketones that may be used in the process according to the invention may be linear, branched or cyclic and generally contain 3, 4, 5, 6, 7, 8, 9 or 10 carbon atoms. Ketones containing 3, 4, 5, 6, 7 or 8 carbon atoms are suitable for use. Among the ketones, acetone, 2-butanone, 2- or 3-pentanone, methyl isobutyl ketone, diisopropyl ketone, cyclohexanone and acetophenone are preferred. Methyl isobutyl ketone is particularly preferred.

The ethers that may be used in the process according to the invention may be linear, branched or cyclic and generally contain 2, 3, 4, 5, 6, 7, 8, 9 or 10 carbon atoms. Ethers containing 4, 5, 6, 7, 8 or 9 carbon atoms are suitable for use. Among the aliphatic or alicyclic ethers, diethyl ether, methyl isopropyl ether, diethylene glycol monomethyl ether, diethylene glycol dimethyl ether, tetrahydrofuran and 1,4-dioxane are preferred.

Where appropriate, the content of non-fluoro organic solvent in the extraction medium may be chosen as a function of the desired polarity of the composition. Generally, this content is not more than 20% by weight Preferably, it is not more than 10% by weight When a non-fluoro organic solvent is present, its content is generally at least 1% by weight Preferably, it is at least 2% by weight.

A first particular example of an extraction medium contains a fluoro compound as described above and ethanol, preferably in an amount as described above.

A second particular example of an extraction medium contains a fluoro compound as described above and n-pentane, preferably in an amount as described above.

A third particular example of an extraction medium contains a fluoro compound as described above and n-hexane, preferably in an amount as described above.

A fourth particular example of an extraction medium consists essentially of 1,1,1,3,3-pentafluorobutane.

A fifth particular example of an extraction medium comprises a fluoro compound, in particular 1,1,1,3,3-pentafluorobutane and a non-fluoro cosolvent in proportions in which they form an azeotrope or pseudo-azeotrope, for example as described above. It has been found that this particular extraction medium allows particularly efficient separation of the organic compound, for example by evaporation of the extraction medium. The extraction medium may be readily recycled. The organic compound may be recovered with a minimum or even nonexistent residual content of extraction medium.

The extraction medium optionally contains a surfactant. Any surfactant that is well known per se and compatible with the extraction medium may be used The surfactant can, for example, improve the wettability of solid substrates.

In the process according to the invention, the treatment with the extraction medium is generally performed at a temperature of greater than or equal to 0° C. Often, this temperature is greater than or equal to 20° C. Preferably, it is greater than or equal to 30° C. In the process according to the invention, the treatment with the extraction medium is generally performed at a temperature of less than or equal to 200° C. Often, this temperature is less than or equal to 100° C. Preferably, it is less than or equal to 80° C. In a particularly preferred manner, it is less than or equal to 70° C.

In the process according to the invention, the treatment with the extraction medium is preferably performed at a pressure of greater than or equal to about 101.3 kPa (1 bar). Often, the pressure is less than or equal to 20 bar. Preferably, it is less than or equal to 10 bar.

In the process according to the invention, the organic compound may be chosen, for example, from oxygen-containing hydrocarbons, nitrogen-containing hydrocarbons or unsaturated hydrocarbons.

Often, the organic compound is a natural product chosen, for example, from terpenes, steroids, triglycerides, saturated or unsaturated fatty acids, prostaglandins, alkaloids and vitamins and also from derivatives of these natural products, in particular oxygen-containing derivatives.

Examples of terpenes that may be mentioned include mono-, sesqui-, di-, tri- and tetraterpenes and derivatives thereof, in particular oxygen-containing derivatives such as alcohols or esters, in particular acetates.

Specific examples of such terpenes that may be separated out, for example, in particular with perfluorohexane, are chosen from β-pinene, limonene, linalool, eugenol, menthol, thymol and linalyl acetate. The said terpenes may also be separated out in particular with 1,1,1,3,3-pentafluorobutane.

The process according to the invention is particularly suitable for the separation of terpene hydrocarbons, for instance α-pinene, β-pinene, limonene, cymene, camphene, sabinene, 3-carene, terpinene, lyrcene, myrcene, t-caryophyllene, squalene and squalane.

The process according to the invention is also suitable for the separation of oxygen-containing terpene hydrocarbons, for instance cineole, carvone, linalool, eugenol, menthol, thymol, linalyl acetate, carvacrol, citral, anethole, terpineol, borneol, camphor, eucalyptol, verbenone, caryophyllene oxide and bomyl acetate.

Specific examples of steroids are chosen, for example, from derivatives comprising a cyclopentaphenanthrene skeleton optionally comprising alkyl and/or oxygen-containing substituents and also, optionally, double bonds. Specific steroids that may be mentioned include β-sitosterol, campesterol, stigmasterol, Δ5-avenasterol, clerosterol, chlosterol, 24-methylene cholesterol, Δ5,23-stigmastadienol, le Δ5,24-stigmastadienol, brassicasterol, oestradiol, oestrogene and testosterone.

Examples of fatty acids that may be mentioned include fatty acids containing at least 6 and preferably at least 8 carbon atoms. Generally, the fatty acids contain not more than 30 and preferably not more than 20 carbon atoms.

Specific examples are chosen from caprylic acid, capric acid, lauric acid, myristic acid, palmitic acid, stearic acid, oleic acid, linoleic acid and linolenic acid.

In the process according to the invention, the substrate is often of natural origin.

In this case, the process according to the invention may be performed, for example, in the following manner:
(a) the substrate of natural origin is prepared, for example by chopping it or micronizing it;
(b) the prepared substrate is treated according to the process according to the invention;
(c) at least some of the fraction comprising organic compound and fluoro compound is recovered;
(d) the recovered portion is subjected to at least one purification operation.

The purification operation may be, for example, an evaporation of at least some of the fluoro compound, a crystallization or a chromatography operation.

It has been found that the fluoro compounds described above, in particular 1,1,1,3,3-pentafluorobutane, may be used as solvent for dewaxing concretes, i.e. for particularly efficiently precipitating waxes from a natural extract, for example from essential oils.

The concretes may be obtained, for example, by extraction of plants containing essential oils with a non-fluoro solvent as described above, in particular hydrocarbons. For the dewaxing, fluorinated organic compound is added to the concrete, which has optionally been concentrated beforehand.

The dewaxing temperature is generally less than or equal to 30° C. Preferably, it is less than or equal to about 25° C. The dewaxing temperature is generally greater than or equal to −10° C. Preferably, it is greater than or equal to about 0° C.

The dewaxing may be performed in two stages, for example first at a temperature of 20 to 30° C. and then at a temperature of 0 to 10° C.

In a first embodiment, the substrate of natural origin is obtained by processing plants. The substrate may, for example, comprise plant leaves, needles or bark.

In a second embodiment, the substrate is obtained by processing materials of animal origin.

The invention also relates to a process for manufacturing a pharmaceutical or cosmetic product containing an organic compound, comprising the separation of the organic compound according to the separation process according to the invention.

The manufacturing process according to the invention is particularly preferably applied when the organic compound is an active principle included in the composition of the pharmaceutical or cosmetic product.

The invention consequently also relates to the use of an extraction medium in accordance with that described above, as an excipient for a pharmaceutical or cosmetic product.

The invention also relates to a pharmaceutical product comprising
(a) an organic compound as active principle
(b) an excipient comprising at least one fluoro compound with an atmospheric boiling point of greater than or equal to 25° C.

The invention also relates to a cosmetic product comprising
(a) an organic compound as active principle
(b) an excipient comprising at least one fluoro compound with an atmospheric boiling point of greater than or equal to 25° C.

The cosmetic product is preferably a fragrance or a cream It has been found that the presence of the fluoro compound creates a pleasant sensation of S freshness when the cosmetic product is placed in contact with the skin.

The invention also relates to a process for preparing a sample intended for the analysis of at least one organic compound from a substrate containing the said organic compound, comprising treatment of the substrate with an extraction medium comprising at least one fluoro compound that has an atmospheric boiling point of greater than or equal to 25° C., so as to form a fraction comprising organic compound and fluoro compound.

A preferred substrate to be processed in the preparation process according to the invention is an aqueous fraction containing traces of plant-protection products, namely pesticides, as organic compound.

It is understood that the teaching and preferences relating to the fluoro compound, and, where appropriate, the extraction medium and its composition, given above in the context of the separation process according to the invention apply in the same manner to the manufacturing process, the uses, the products and the preparation process described above.

The examples below are intended to illustrate the invention without, however, limiting it.

### EXAMPLE 1

Chopped fresh rosemary leaves were subjected to treatment with pure 1,1,1,3,3-pentafluorobutane. The pressure was atmospheric pressure and the process was performed at reflux for a period of 1 hour. The mass ratio between the 1,1,1,3,3-pentafluorobutane and the rosemary leaves was 4.8. The mixture was filtered while hot and a faction of 1,1,1,3,3-pentafluorobutane containing compounds extracted from the rosemary was recovered, including 89.4% of terpenes having the following composition:

| Compound | relative % |
|---|---|
| Camphor | 29.48 |
| Eucalyptol | 20.08 |
| Borneol | 9.75 |
| α-Pinene | 4.34 |
| Camphene | 3.73 |
| α-Terpineol | 2.59 |
| Limonene | 2.26 |
| β-Pinene | 2.48 |
| Verbenone | 3.15 |
| β-Myrcene | 0.71 |
| Cymene | 1.39 |
| γ-Terpinene | 0.93 |
| Terpinolene | 0.59 |
| 3-Carene | 0.94 |
| Caryophyllene oxide | 1.29 |
| Caryophyllene | 3.80 |
| Linalool | 1.87 |

The aromatic profile obtained was close to the composition of natural essential oil of rosemary.

### EXAMPLE 2

Chopped fresh Serenoa repens fruit was subjected to three successive treatments with pure 1,1,1,3,3-pentafluorobutane. The pressure was atmospheric pressure and the process was performed at reflux for a period of 30 minutes for each treatment. The respective mass ratio between the 1,1,1,3,3-pentafluoro-10 butane and the fruit was 6.3. The mixture was filtered and a combined fraction of 1,1,1,3,3-pentafluorobutane was recovered containing 9.38% of extracted compounds, including 0.45% of steroids having the following composition:

| Compound | relative % |
|---|---|
| β-Sitosterol | 66.5 |
| Campesterol | 18.4 |
| Stigmasterol | 8.3 |
| Δ5-Avenasterol | 3.0 |
| Clerosterol | 1.0 |
| Chlolesterol | 0.9 |
| 24-Methylene cholesterol | 0.9 |
| Δ5.23-Stigmastadienol | 0.1 |
| Δ5.24-Stigmastadienol | 0.4 |
| Brassicasterol | 0.1 |

The 1,1,1,3,3-pentafluorobutane faction also contained fatty acids having the following composition:

| Acid | % |
|---|---|
| caprylic | 1.1 |
| capric | 2.5 |
| lauric | 27.4 |
| myristic | 11.7 |
| palmitic | 10.0 |
| stearic | 2.3 |
| oleic | 29.3 |
| linoleic | 6.6 |
| linolenic | 0.8 |

1- Process for separating out at least one organic compound from a substrate containing the said organic compound, comprising a treatment of the substrate with an extraction medium comprising at least one fluoro compound with an atmospheric boiling point of greater than or equal to 25° C., so as to form a fraction comprising organic compound and fluoro compound.

2- Process according to claim 1, in which the fluoro compound contains only fluorine as halogen.

3- Process according to claim 1 or 2, in which the fluoro compound is chosen from fluoro ethers, hydrofluoroalkanes and perfluoroalkanes.

4- Process according to any one of claims 1 to 3, in which the fluoro compound has an atmospheric boiling point of from 30 to 80° C.

5- Process according to any one of claims 1 to 4, in which the fluoro compound is chosen from 1,1,1,3,3-pentafluorobutane, perfluorobutyl methyl ether, perfluorobutyl ethyl ether, 1,1,1,2,3,4,4,5,5,5-decafluoropentane, the H-Galden® hydrofluoropolyethers and the Galdel® perfluoropolyethers, perfluoropentane and perfluorohexane.

6- Process according to claim 5, in which the fluoro compound is 1,1,1,3,3-pentafluorobutane.

7- Process according to any one of claims 1 to 5, in which the extraction medium also comprises a non-fluoro cosolvent.

8- Process according to claim 7, in which the extraction medium comprises a hydrocarbon, a dialkyl ether or an alkanol as cosolvent

9- Process according to claim 8, in which the extraction medium comprises an azeotropic or pseudo-azeotropic composition of 1,1,1,3,3-pentafluorobutane and ethanol.

10- Process according to claim 8, in which the exaction medium comprises an azeotropic or pseudo-azeotropic composition of 1,1,1,3,3-pentafluorobutane and n-hexane.

11- Process according to any one of claims 1 to 10, in which the organic compound is chosen from oxygen-containing hydrocarbons, nitrogen-containing hydrocarbons and unsaturated hydrocarbons.

12- Process according to any one of claims 1 to 11, in which the organic compound is chosen from terpenes, steroids, prostaglandins, alkaloids, vitamins and derivatives thereof, in particular oxygen-containing derivatives thereof

13- Process according to any one of claims 1 to 12, in which the substrate is of natural origin.

14- Process according to claim 13, in which the substrate has been obtained by processing plants.

15- Process according to claim 13, in which the substrate has been obtained by processing materials of animal origin.

16- Process according to any one of claims 1 to 15, in which the processing is performed at a temperature of from 20 to 200° C.

17- Process according to any one of claims 1 to 16, in which the processing is performed at a pressure of from 1 to 20 bar.

18- Process for manufacturing a pharmaceutical or cosmetic product containing an organic compound, comprising the separation of the organic compound according to the process of any one of claims 1 to 17.

19- Use of an extraction medium in accordance with any one of claims 1 to 10, as an excipient for a pharmaceutical or cosmetic product

20- Pharmaceutical product comprising
a) an organic compound as active principle
b) an excipient comprising at least one fluoro compound with an atmospheric boiling point of greater than or equal to 25° C.

21- Cosmetic product comprising
a) an organic compound as active principle
b) an excipient comprising at least one fluoro compound that has an atmospheric boiling point of greater than or equal to 25° C.

22- Process for preparing a sample intended for the analysis of at least one organic compound from a substrate containing the said organic compound, comprising treatment of the substrate with an extraction medium comprising at least one fluoro compound that has an atmospheric boiling point of greater than or equal to 20° C., so as to form a fraction comprising organic compound and fluoro compound

23- Use of a fluoro compound with an atmospheric boiling point of greater than or equal to 25° C., as a solvent for dewaxing concretes.

24- Use, product or process according to any one of claims 20 to 23, in which the fluoro compound is 1,1,1,3,3-pentafluorobutane.
